(19) United States
(12) Patent Application Publication
(10) Pub. No.: US 2025/0359847 A1
(43) Pub. Date: Nov. 27, 2025

(54) ULTRASOUND AUXILIARY DEVICE, AND ULTRASOUND PROBE INCLUDING THE SAME

(71) Applicant: HUKUI BIOTECHNOLOGY CO., LTD., Taipei City (TW)

(72) Inventors: SHIH-CHE LO, Taipei City (TW); CHUN-CING CHEN, Taipei City (TW)

(73) Assignee: HUKUI BIOTECHNOLOGY CO., LTD., Taipei City (TW)

(21) Appl. No.: 19/216,735

(22) Filed: May 23, 2025

Related U.S. Application Data
(60) Provisional application No. 63/651,412, filed on May 24, 2024.

(30) Foreign Application Priority Data
Aug. 5, 2024 (TW) 113129165

(51) Int. Cl. A61B 8/00

(52) U.S. Cl. CPC A61B 8/4209; A61B 8/4281

(57) ABSTRACT

An ultrasound auxiliary device, comprising: a main body, including: a cavity, located inside the main body, wherein the cavity can be used to contain fluid, and the ultrasound auxiliary device is configured to adjust a thickness thereof according to a volume or distribution of the fluid located inside the cavity; and a plurality of positioning marks, located in the main body, wherein the positioning marks are arranged in at least two directions perpendicular to each other, and the positioning marks have the same spacing in any of the directions.

### CROSS-REFERENCE TO RELATED APPLICATION

This non-provisional application claims priority under 35 U.S.C. § 119 (a) on Patent Application No(s). 113129165 filed in Taiwan, R.O.C. on Aug. 5, 2024, the entire contents of which are hereby incorporated by reference.

This non-provisional application claims priority under 35 U.S.C. § 119 (e) on US provisional Patent Application No(s). 63/651,412 filed on May 24, 2024, the entire contents of which are hereby incorporated by reference.

### BACKGROUND OF THE INVENTION

### 1. Field of the Invention

The present disclosure relates to an ultrasound auxiliary device, and in particular to an ultrasound auxiliary device including a cavity. The present disclosure also relates to an ultrasound probe including the ultrasound auxiliary device.

### 2. Description of the Related Art

Ultrasound imaging technology is widely used in the diagnosis of human bodies. An ultrasound generator in an ultrasound probe is composed of many small units, and ultrasonic waves generated by these small units will interfere with each other within a certain distance, resulting in poor image quality of an ultrasound image. A region where interference occurs is generally called near field. Beyond a certain distance, the ultrasonic waves generated by these small units will not interfere with each other. Beyond this distance, ultrasonic energy generated by these small units is evenly distributed, and its waveform is similar to that of an acoustic wave generated by a single point source of wave. Therefore, beyond this distance, the image quality of the ultrasound image is good. In contrast to near field, a region where no interference occurs is generally referred to as far field.

Due to the above-mentioned characteristics, a good imaging quality can be obtained only by keeping a specific distance between the ultrasound probe and a target region to be imaged to locate the target region in the far field. However, due to the differences in the depth of different organs in a human body, it is often difficult to make a single probe suitable for the diagnosis of all organs in the human body.

### BRIEF SUMMARY OF THE INVENTION

There is still room for improvement in conventional ultrasound imaging technology. Therefore, one of the objectives of the present disclosure is to provide an ultrasound auxiliary device, which can adjust a distance between an ultrasound probe and a target region to be imaged, so as to improve the imaging quality, and when sampling is required, the accuracy of sampling can be improved.

In order to achieve the above-mentioned objectives and other objectives, the present disclosure provides an ultrasound auxiliary device, comprising: a main body, including:
a cavity, located inside the main body, wherein the cavity can be used to contain fluid, and the ultrasound auxiliary device is configured to adjust a thickness thereof according to a volume or distribution of the fluid located inside the cavity; and
a plurality of positioning marks, located in the main body, wherein the positioning marks are arranged in at least two directions perpendicular to each other, and the positioning marks have the same spacing in any of the directions.

In an embodiment of the present disclosure, the main body further includes:
an injection port, located on the main body's side wall and in fluid communication with the cavity;
an exhaust port, located on the main body's side wall and in fluid communication with the cavity; and
a plug, closing the exhaust port.

In an embodiment of the present disclosure, the main body is composed of silicone, polyurethane, or rubber.

In order to achieve the above-mentioned objectives and other objectives, the present disclosure also provides an ultrasound probe, including an ultrasound auxiliary device as described above at a front end thereof.

In an embodiment of the present disclosure, the ultrasound probe may include a capacitive micromachined ultrasonic transducer (CMUT) or a piezoelectric micromachined ultrasonic transducer (PMUT).

According to the ultrasound auxiliary device and the ultrasound probe of the present disclosure, by setting the cavity, the distance between the ultrasound probe and the target region to be imaged can be adjusted, so as to improve the imaging quality, and when sampling is required, a sampling device can be guided by means of the plurality of positioning marks, so as to improve the sampling accuracy.

### BRIEF DESCRIPTION OF THE DRAWINGS

For the sake of clarity, the thickness or dimensions of each layer in the drawings are depicted in an enlarged, omitted, or schematic manner. At the same time, the dimensions of each element do not necessarily reflect their actual dimensions.

FIG. 1 is a schematic cross-sectional view of an ultrasound auxiliary device in Embodiment 1 of the present disclosure.

FIG. 2 is a schematic cross-sectional view of the ultrasound auxiliary device in a first state of use in Embodiment 1 of the present disclosure.

FIG. 3 is a schematic cross-sectional view of the ultrasound auxiliary device in a second state of use in Embodiment 1 of the present disclosure.

FIG. 4 is a schematic cross-sectional view of an ultrasound auxiliary device in Embodiment 2 of the present disclosure.

FIG. 5 is a schematic cross-sectional view of the ultrasound auxiliary device in a state of use in Embodiment 2 of the present disclosure.

FIG. 6 is a schematic cross-sectional view of an ultrasound probe in Embodiment 3 of the present disclosure.

### DETAILED DESCRIPTION OF THE INVENTION

In order to fully understand the purposes, features and effects of the present disclosure, the present disclosure is described below in detail with the following specific embodiments:

### Embodiment 1

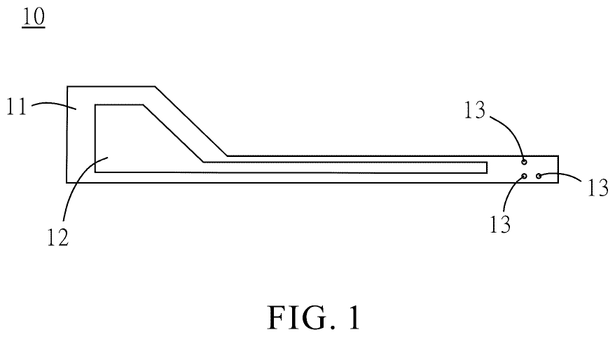

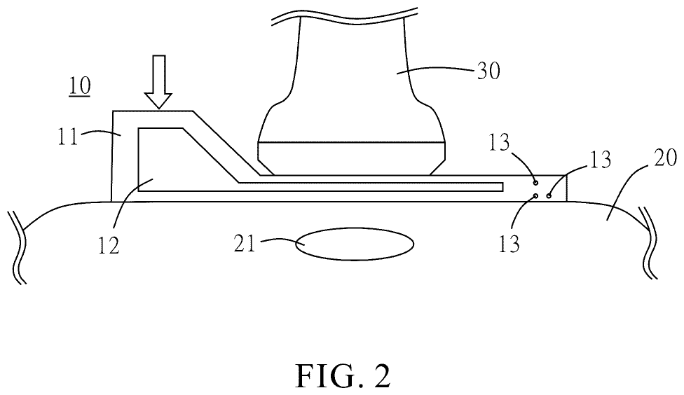

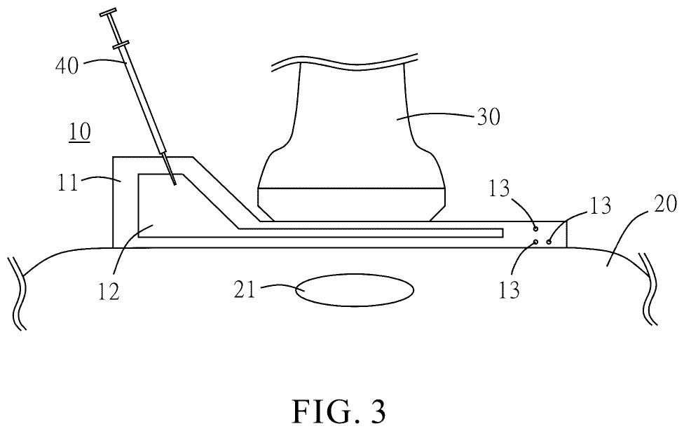

An ultrasound auxiliary device in Embodiment 1 of the present disclosure is as shown in FIG. 1, FIG. 2 and FIG. 3.

The ultrasound auxiliary device 10 in Embodiment 1 of the present disclosure comprises: a main body 11. By way of example, the main body 11 may be composed of silicone, polyurethane, or rubber, but the present disclosure is not limited to this.

The main body 11 includes: a cavity 12, located inside the main body 11, wherein the cavity 12 can be used to contain fluid, and the ultrasound auxiliary device 10 is configured to adjust a thickness thereof according to a volume or distribution of the fluid located inside the cavity 12.

The main body 11 also includes: a plurality of positioning marks 13, located in the main body 11, wherein the positioning marks 13 are arranged in at least two directions perpendicular to each other, and the positioning marks 13 have the same spacing in any of the directions. Preferably, there is a considerable difference in the ultrasonic transmission properties between the plurality of positioning marks 13 and the main body 11, so that the plurality of positioning marks 13 can be clearly imaged with ultrasound. By way of example, a material with a density significantly higher than that of the main body 11 can be selected to form the plurality of positioning marks 13.

As shown in FIG. 2, in a first state of use, when the ultrasound auxiliary device 10 in Embodiment 1 of the present disclosure is in use, the cavity 12 contains fluid, wherein the fluid may be water, normal saline or gel, but the present disclosure is not limited to this.

As shown in FIG. 2, in the first state of use, a lower surface of the ultrasound auxiliary device 10 in Embodiment 1 of the present disclosure is attached to a human body 20, thereby making a surface of the human body 20 smooth and thus improving the convenience of operation. By way of example, when it is desired to acquire an ultrasound image of the thyroid gland located in the throat, the ultrasound auxiliary device 10 in Embodiment 1 of the present disclosure can be used to make the throat smooth, thereby avoiding unexpected sliding of an ultrasound probe 30 during operation due to uneven surface of the throat.

As shown in FIG. 2, in the first state of use, when the ultrasound auxiliary device 10 in Embodiment 1 of the present disclosure is in use, the ultrasound probe 30 is attached to an upper surface of the ultrasound auxiliary device 10.

As shown in FIG. 2, in the first state of use, the ultrasound auxiliary device 10 in Embodiment 1 of the present disclosure can keep a certain distance between the ultrasound probe 30 and a lesion 21, and a user can change the pressure applied to the position of the arrow shown in FIG. 2 as needed to adjust the distance between the ultrasound probe 30 and the lesion 21 in real time.

By way of example, when the lesion 21 is located in a relatively shallow region of the human body 20 (for example, located at the thyroid gland in the throat), larger pressure can be applied to the position of the arrow shown in FIG. 2. Due to the application of the larger pressure, more fluid will flow from a region below the arrow shown in FIG. 2 to a region below the ultrasound probe 30, so that the thickness of the ultrasound auxiliary device 10 at the ultrasound probe 30 is increased. The larger thickness can keep the ultrasound probe 30 at a longer distance from the human body 20, which can avoid that imaging is poor due to interference caused by the fact that the ultrasound probe 30 and the lesion 21 are too close to make the lesion 21 located in near field when the lesion 21 is located in the relatively shallow region of the human body 20 (for example, located at the thyroid gland in the throat).

In contrast, when the lesion 21 is located in a relatively deep region of the human body 20 (for example, located at an abscess in the liver), less pressure can be applied to the position of the arrow shown in FIG. 2. Due to the application of the less pressure, more fluid remains in the region below the arrow shown in FIG. 2, so that the ultrasound auxiliary device 10 has a smaller thickness at the ultrasound probe 30. The smaller thickness can keep the ultrasound probe 30 closer to the human body 20, which can avoid that imaging is poor due to the fact that the distance between the ultrasound probe 30 and the lesion 21 is too long when the lesion 21 is located in the relatively deep region of the human body 20 (for example, located at the abscess in the liver).

As shown in FIG. 3, in a second state of use, when the ultrasound auxiliary device 10 in Embodiment 1 of the present disclosure is in use, the cavity 12 contains fluid, wherein the fluid is injected into the cavity 12 by an injector 40, and a user can adjust the distance between the ultrasound probe 30 and the lesion 21 in real time by adjusting the volume of the injected fluid.

In a preferred embodiment, the main body 11 is composed of a material (e.g., silicone, polyurethane or rubber) that can self-repair after being punctured by a needle of the injector 40. Therefore, when the volume of the fluid in the cavity 12 reaches the demand, the injector 40 can be pulled out, and after the injector 40 is pulled out, a needle hole formed by the injector 40 can self-repair due to the elasticity of the material that constitutes the main body 11, so that the fluid in the cavity 12 will not flow out from the needle hole.

As shown in FIG. 2 and FIG. 3, an operator can simultaneously observe the plurality of positioning marks 13 and the lesion 21 from the ultrasound image to determine the relative distance between the plurality of positioning marks 13 and the lesion 21. In this way, when the lesion 21 needs to be sampled, the operator can, with the plurality of positioning marks 13, determine the position and angle at which the sampling device inserts a needle into the surface of the human body 20, thereby improving the accuracy of sampling.

By way of example, the ultrasound auxiliary device 10 in Embodiment 1 of the present disclosure can be applied to thyroid ultrasound, breast ultrasound or liver ultrasound and sampling of related lesions, but the present disclosure is not limited to this.

### Embodiment 2

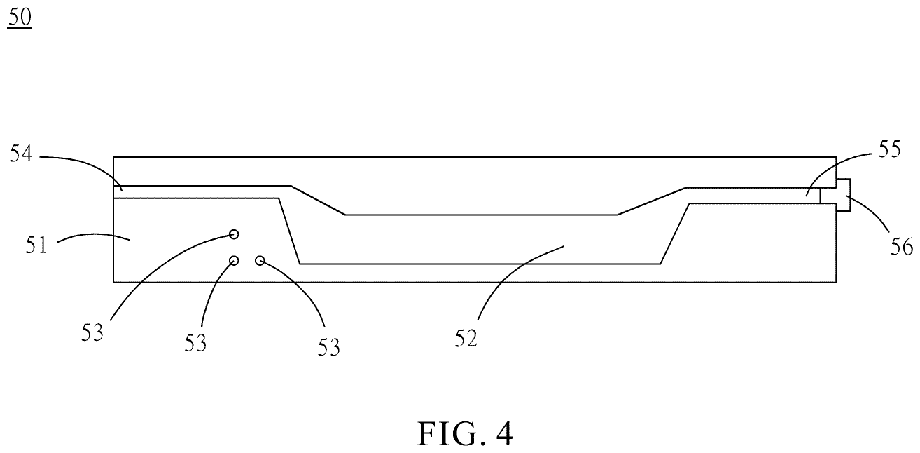

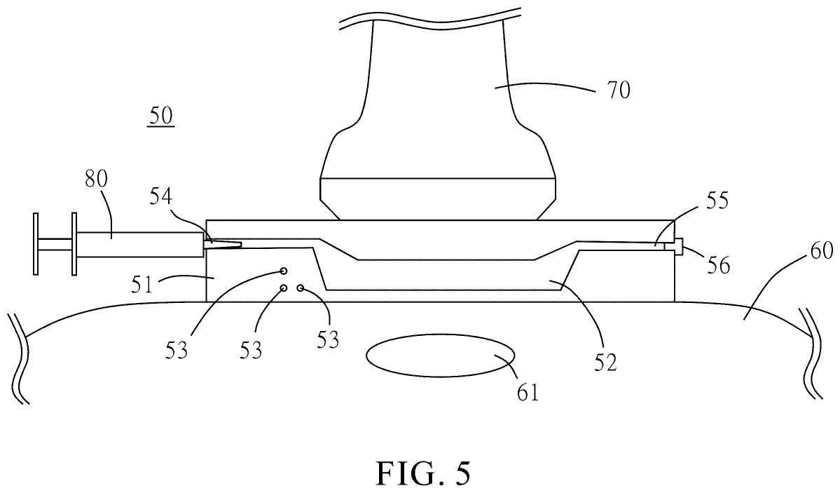

An ultrasound auxiliary device in Embodiment 2 of the present disclosure is as shown in FIG. 4 and FIG. 5.

The ultrasound auxiliary device 50 in Embodiment 2 of the present disclosure comprises: a main body 51. By way of example, the main body 51 may be composed of silicone, polyurethane, or rubber, but the present disclosure is not limited to this.

The main body 51 includes: a cavity 52, located inside the main body 51, wherein the cavity 52 can be used to contain fluid, and the ultrasound auxiliary device 50 is configured to adjust a thickness thereof according to a volume of the fluid located inside the cavity 52.

The main body 51 also includes: a plurality of positioning marks 53, located in the main body 51, wherein the positioning marks 53 are arranged in at least two directions perpendicular to each other, and the positioning marks 53 have the same spacing in any of the directions. Preferably, there is a considerable difference in the ultrasonic transmission properties between the plurality of positioning marks 53 and the main body 51, so that the plurality of positioning marks 53 can be clearly imaged with ultrasound. By way of example, a material with a density significantly higher than that of the main body 51 can be selected to form the plurality of positioning marks 53.

Compared with Embodiment 1, the main body 51 of the ultrasound auxiliary device 50 in Embodiment 2 of the present disclosure further includes: an injection port 54, located on the main body's side wall and in fluid communication with the cavity 52; an exhaust port 55, located on the main body's side wall and in fluid communication with the cavity 52; and a plug 56, closing the exhaust port 55.

As shown in FIG. 5, in a state of use, when the ultrasound auxiliary device 50 in Embodiment 2 of the present disclosure is in use, the cavity 52 contains fluid, wherein the fluid may be water, normal saline or gel, but the present disclosure is not limited to this.

As shown in FIG. 5, in the state of use, a lower surface of the ultrasound auxiliary device 50 in Embodiment 2 of the present disclosure is attached to a human body 60, thereby making a surface of the human body 60 smooth and thus improving the convenience of operation. By way of example, when it is desired to acquire an ultrasound image of the thyroid gland located in the throat, the ultrasound auxiliary device 50 in Embodiment 2 of the present disclosure can be used to make the throat smooth, thereby avoiding unexpected sliding of an ultrasound probe 70 during operation due to uneven surface of the throat.

As shown in FIG. 5, in the state of use, when the ultrasound auxiliary device 50 in Embodiment 2 of the present disclosure is in use, the ultrasound probe 70 is attached to an upper surface of the ultrasound auxiliary device 50.

As shown in FIG. 5, in the state of use, when the ultrasound auxiliary device 50 in Embodiment 2 of the present disclosure is in use, the cavity 52 contains fluid, wherein the fluid is injected into the cavity 52 through the injection port 54 by an injector 80, and when the fluid is injected, the plug 56 can be pulled out, so that gas in the cavity 52 can be exhausted through the exhaust port 55. After the gas is exhausted, the plug 56 can be plugged back into the exhaust port 55 to avoid that the fluid leaks from the exhaust port 55.

As shown in FIG. 5, the ultrasound auxiliary device 50 in Embodiment 2 of the present disclosure can keep a certain distance between the ultrasound probe 70 and a lesion 61, and a user can adjust the distance between the ultrasound probe 70 and the lesion 61 in real time by adjusting the volume of the injected fluid.

By way of example, when the lesion 61 is located in a relatively shallow region of the human body 60 (for example, located at the thyroid gland in the throat), more fluid can be injected. Since more fluid is injected, the ultrasound auxiliary device 50 will have a larger thickness. The larger thickness can keep the ultrasound probe 70 at a longer distance from the human body 60, which can avoid that imaging is poor due to interference caused by the fact that the ultrasound probe 70 and the lesion 61 are too close to make the lesion 61 located in near field when the lesion 61 is located in the relatively shallow region of the human body 60 (for example, located at the thyroid gland in the throat).

In contrast, when the lesion 61 is located in a relatively deep region of the human body 60 (for example, located at an abscess in the liver), less fluid can be injected. Since less fluid is injected, the ultrasound auxiliary device 50 will have a smaller thickness. The smaller thickness can keep the ultrasound probe 70 closer to the human body 60, which can avoid that imaging is poor due to the fact that the distance between the ultrasound probe 70 and the lesion 61 is too long when the lesion 61 is located in the relatively deep region of the human body 60 (for example, located at the abscess in the liver).

As shown in FIG. 5, an operator can simultaneously observe the plurality of positioning marks 53 and the lesion 61 from the ultrasound image to determine the relative distance between the plurality of positioning marks 53 and the lesion 61. In this way, when the lesion 61 needs to be sampled, the operator can, with the plurality of positioning marks 53, determine the position and angle at which the sampling device inserts a needle into the surface of the human body 60, thereby improving the accuracy of sampling.

### Embodiment 3

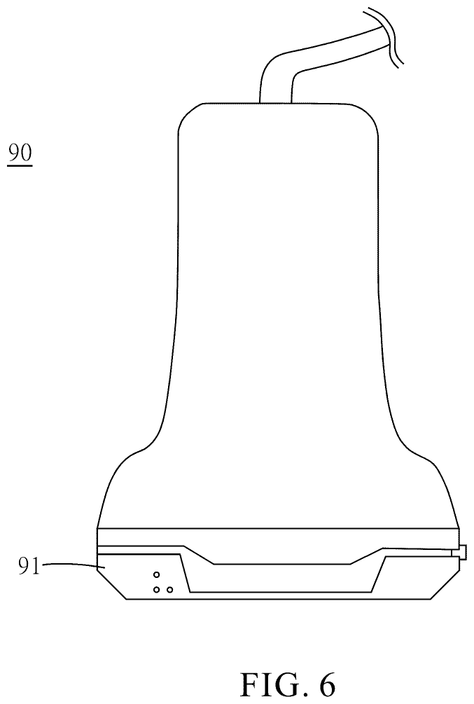

An ultrasound probe in Embodiment 3 of the present disclosure is as shown in FIG. 6.

The ultrasound probe 90 in Embodiment 3 of the present disclosure includes an ultrasound auxiliary device 91 as described in Embodiment 2 at a front end thereof. By way of example, the ultrasound probe 90 includes a capacitive micromachined ultrasonic transducer (CMUT) or a piezoelectric micromachined ultrasonic transducer (PMUT), but the present disclosure is not limited to this.

According to the ultrasound probe 90 in Embodiment 3 of the present disclosure, the ultrasound auxiliary device 91 as described in Embodiment 2 is integrated at the front end of the probe. Therefore, a distance between the ultrasound probe and a target region to be imaged can be adjusted by means of a cavity of the ultrasound auxiliary device 91, so as to improve the imaging quality, and when sampling is required, the accuracy of sampling can be improved by means of positioning marks of the ultrasound auxiliary device 91.

The present disclosure has been disclosed in the preferred embodiments hereinabove, but those skilled in the art should understand that these embodiments are only used to illustrate the present disclosure and should not be interpreted as limiting the scope of the present disclosure. It should be noted that all changes and substitutions equivalent to the embodiments should be considered to fall within the scope of the present disclosure. Therefore, the scope of protection of the present disclosure shall be defined by the claims.

While the present disclosure has been described by means of specific embodiments, numerous modifications and variations could be made thereto by those skilled in the art without departing from the scope and spirit of the present disclosure set forth in the claims.

What is claimed is:

1. An ultrasound auxiliary device, comprising:
a main body, including:
a cavity, located inside the main body, wherein the cavity can be used to contain fluid, and the ultrasound auxiliary device is configured to adjust a thickness thereof according to a volume or distribution of the fluid located inside the cavity; and
a plurality of positioning marks, located in the main body, wherein the positioning marks are arranged in at least two directions perpendicular to each other, and the positioning marks have the same spacing in any of the directions.

2. The ultrasound auxiliary device according to claim 1, wherein the main body further comprises:
an injection port, located on the main body's side wall and in fluid communication with the cavity;
an exhaust port, located on the main body's side wall and in fluid communication with the cavity; and
a plug, closing the exhaust port.

3. The ultrasound auxiliary device according to claim 1, wherein the main body is composed of silicone, polyurethane, or rubber.

4. An ultrasound probe, including an ultrasound auxiliary device according to claim 1 at a front end thereof.

5. The ultrasound probe according to claim 4, wherein the ultrasound probe includes a capacitive micromachined ultrasound transducer or a piezoelectric micromachined ultrasonic transducer.
